(19) United States
(12) Patent Application Publication
(10) Pub. No.: US 2024/0208893 A1
(43) Pub. Date: Jun. 27, 2024

(54) CONTINUOUS PROCESS FOR THE PRODUCTION OF AMINES IN THE GAS PHASE USING A RECYCLE GAS MODE

(71) Applicant: BASF SE, Ludwigshafen am Rhein (DE)

(72) Inventors: Tatjana HUBER, Ludwigshafen am Rhein (DE); Joerg PASTRE, Ludwigshafen am Rhein (DE); Johann-Peter MELDER, Ludwigshafen am Rhein (DE); Thomas KRUG, Ludwigshafen am Rhein (DE); Kristin SCHROEDER, Florham Park, NJ (US)

(21) Appl. No.: 18/286,582

(22) PCT Filed: Mar. 30, 2022
(86) PCT No.: PCT/EP2022/058403

§ 371 Date: Oct. 12, 2023

(30) Foreign Application Priority Data
Apr. 14, 2021 (EP) 21168407.1
Nov. 22, 2021 (EP) 21209625.9

(51) Int. Cl. C07C 209/16; B01J 21/04; B01J 23/72; B01J 23/755; C07C 213/02; C07D 207/06; C07D 403/06

(52) U.S. Cl. CPC C07C 209/16; B01J 21/04; B01J 23/72; B01J 23/755; C07C 213/02; C07D 207/06; C07D 403/06

(57) ABSTRACT

A process for the continuous production of amines, the process comprising reacting a primary or secondary alcohol with ammonia in the presence of hydrogen and a heterogeneous hydrogenation catalyst in the gas phase using a recycle gas mode, wherein the temperature in the pressure separator is >20° C.

### DESCRIPTION

This invention relates to a process for the continuous production of amines, the process comprising reacting a primary or secondary alcohol with ammonia in the presence of hydrogen and a heterogeneous hydrogenation catalyst in the gas phase using a recycle gas mode.

### STATE OF THE ART

The process products are used, inter alia, as intermediates in the production of fuel additives (U.S. Pat. No. 3,275,554; DE-A-21 25 039 and DE-A-36 11 230), surfactants, drugs and crop protection agents, hardeners for epoxy resins, catalysts for polyurethanes, intermediates for the preparation of quaternary ammonium compounds, plasticizers, corrosion inhibitors, synthetic resins, ion exchangers, textile assistants, dyes, vulcanization accelerators and/or emulsifiers.

WO 2010/031719 A1 relates to a process for the preparation of amines in the gas phase. In the experimental section the production of various amines using a gas recycle mode is taught. According to the examples the reactor output is cooled to 10° C. and fed into a pressure separator (page 22, line 29).

DE 10 2004 023 529 A1 (BASF) relates to a process for the preparation of amines in the gas phase using a heterogeneous catalyst comprising CuO, NiO and Al₂O₃.

EP 70 397 A1 (BASF) relates to a process for the production of cyclic amines. According to the examples the reaction mixture is cooled to 20° C. and fed into a pressure separator (page 6, lines 32 to 34).

DE 199 57 672 A1 (BASF) relates to the production of pyrrolidine and describes the formation of bis(pyrrolidino)butane as a side product and its separation from pyrrolidine.

### TECHNICAL PROBLEM

The technical problem to be solved by the present invention was to improve existing processes for the production of amines from corresponding primary alcohols, and to remedy one or more disadvantages of the prior art. The intention was to find a process to be performed with high conversion, high yields, including space-time yield, and selectivity.

The technical problem was also to find a process for the co-production of pyrrolidine and bis(pyrrolidino)butane to be performed with high conversion, high yields, including space-time yield, and selectivity, including efficient measures for their separation and isolation with high purities.

Surprisingly it has been found that the technical problem as specified above can be solved by a process for the continuous production of amines, the process comprising reacting a primary or secondary alcohol with ammonia in the presence of hydrogen and a heterogeneous hydrogenation catalyst in the gas phase using a recycle gas mode, wherein the temperature in the pressure separator is >20° C.

It was surprising that the temperature in the pressure separator has a significant impact on the selectivity of the desired amine reaction product. Given the fact that the art teaches a temperature in the pressure separator of 10° C. or 20° C., respectively (cf. WO 2010/031719 A1, page 22, line 29 and EP 70 397 A1, page 6, lines 32 to 34), it was surprising that a temperature of >20° C. in the pressure separator leads to an increase in amine selectivity.

### DETAILED DESCRIPTION OF THE INVENTION

Preferably the temperature in the pressure separator is >21° C. or even >25° C. Best results with respect to amine selectivity can be obtained by realizing a temperature in the pressure separator which is >30° C. Preferably the temperature in the pressure separator is in the range from 30 to 70° C., even more preferably from 30 to 60° C.

The process is carried out in a reactor or in a plurality of reactors. Unless explicitly provided otherwise, the term “reactor” also covers a “plurality of reactors”. The recycle gas mode is realized by feeding the reaction mixture obtained in the reactor into a pressure separator, where the reaction mixture is separated into a gaseous stream and a liquid reaction product stream (also referred to as “product stream”) and recycling the gaseous stream to the reactor. Such gaseous stream mainly consists of hydrogen and ammonia (no significant amount of the product amines can be found therein). Usually, a part of such gaseous stream is discharged. Otherwise, the amount of gas to be handled would constantly increase because fresh hydrogen and fresh ammonia being continuously fed to the reaction. That part of the gaseous stream being recycled is also referred to as the “recycle gas” or “recycle gas stream”. The recycle gas stream usually can have a flow rate in the range from 40 to 1500 m³, preferably 100 to 700 m³ (at operating pressure)/[m³ of catalyst (bed volume)·h.

Usually, the temperature in the pressure separator is realized by cooling the reaction mixture leaving the reactor.

The pressure separator is usually operated at a pressure close to the reaction pressure which is further specified below. Usually, the pressure in the pressure separator is 0.01 to 10 bar (for example 0.1 to 10 bar), preferably 0.01 to 5 bar, particularly preferably 0.5 to 3 bar below the reaction pressure.

The product stream obtained in the pressure separator can be fed to a low-pressure separator which is operated at a pressure below the reaction pressure. The typical pressure is within the range from 1 to 10 bar. In the low-pressure separator the remaining amounts of hydrogen and ammonia as well as other low boilers depending on the respective amination reaction are separated off. The resulting liquid stream contains the respective product amine and can be further separated.

It is also possible to feed the product stream obtained in the pressure separator into a distillation column, where the remaining amounts of hydrogen and ammonia as well as other low boilers depending on the respective amination reaction can be separated off. Preferably, the ammonia is recycled to the reaction as fresh ammonia. The resulting liquid stream contains the respective product amine and can be further separated. This is further specified below for the coproduction of pyrrolidine and bis(pyrrolidino)butane.

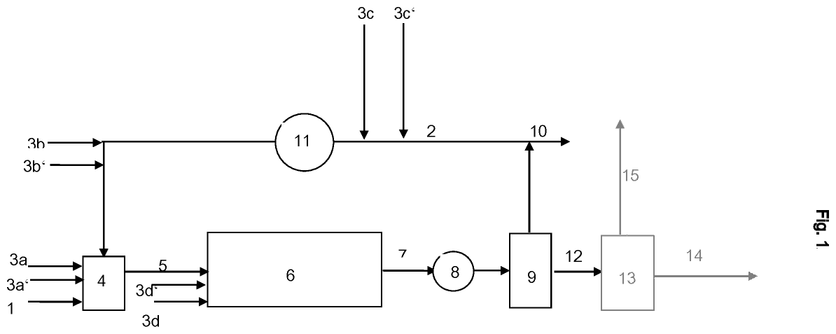

The reaction is carried out in the vapor phase. For that purpose, an evaporator can be used, in which the respective alcohol is evaporated in a gaseous stream, which is usually the recycle gas stream. Fresh hydrogen and ammonia can be fed directly into the evaporator. Moreover, it is also possible to feed fresh hydrogen and/or ammonia directly into the recycle gas stream or into the reactor. A typical set-up for the process according to the present invention is described in FIG. 1.

The process of the invention is carried out continuously, with the catalyst preferably being installed as a fixed bed in the reactor. Flow into the fixed catalyst bed can occur either from above or from below. The temperature, pressure and amount of the gas stream are set so that even relatively high-boiling reaction products remain in the gas phase.

Preferably the reaction is carried out in a tube reactor, particularly a tube-bundle reactor or a single-stream plant. In case of a single-stream plant the tube reactor in which the reaction is carried out preferably consists of a plurality of (e.g. two or three) individual tube reactors connected in series. In case the reaction is carried out in any such reactor(s), any reaction pressure or respective range specified herein refers to the reaction pressure at the reactor inlet. As specified above the pressure in the pressure separator is usually 0.01 to 10 bar (for example 0.1 to 10 bar), preferably 0.01 to 5 bar, particularly preferably 0.5 to 3 bar below the reaction pressure. Any respective drop in pressure thus includes a drop in pressure occurring over the length of the reactor. A further drop in pressure can also occur as a result of cooling the reaction mixture leaving the reactor before entering the pressure separator.

The fresh ammonia is for example added in a molar amount which is from 0.90 to 100, preferably 1 to 30, particularly preferably 1.5 to 10 or even 2 to 8 times that of the alcohol. It is to be understood that these ranges refer to the molar amount of the fresh alcohol that is added to the reaction and neglects any traces of alcohol that might be contained in the recycle gas stream. For the avoidance of doubt, reference is made to the molar amount of entire alcohol molecule; not the molar amount of alcohol functional groups. It is to be noted, that the overall amount of ammonia in the reactor exceeds the amount of fresh ammonia as it results from the amount of fresh ammonia plus the ammonia contained in the recycle gas stream, the latter not being regarded as fresh ammonia. Fresh ammonia can also be any ammonia that is separated off from the product stream, e.g. in an ammonia column, leaving the pressure separator and being recycled.

Fresh hydrogen is usually added in an amount of 100 to 1000, preferably 150 to 550 NL per (volume of catalyst in L and hour) with NL=standard liters=volume converted to STP. STP means standard conditions for temperatures and pressure.

The reaction can be carried out at an absolute pressure in a range from 1 to 300 bar, preferably 10 to 50 bar, particularly preferably 10 to 30 bar or even 15 to 30 bar.

The reaction can be carried out at a temperature in a range from 80 to 300° C., preferably 100 to 250° C., particularly preferably 150 to 240° C. or even 170 to 230° C. The reaction can be carried out adiabatically, isothermally or quasi isothermally (i.e. isoperibolically) provided in each case that the temperature in the reactor is within the respective range as per the preceding sentence. Preferably the reaction is carried out with an isoperibolic temperature profile to control the temperature of the reaction within borders of ±15 K, particularly preferably ±10 K.

These temperature fluctuations are based on the prevailing temperatures in the respective catalyst bed at the point where the starting materials enter the catalyst bed and at the point where the reaction mixtures leave the catalyst bed.

It is possible for a plurality of catalyst beds to be connected in parallel or in series.

If a plurality of catalyst beds are connected in series, the specified temperature fluctuations in the isothermal or isoperibol mode of operation according to the invention apply to the respective temperature in the catalyst bed at the point where the starting materials enter the first catalyst bed and where the reaction mixture leaves the last catalyst bed.

In a preferred embodiment, the temperature of the reactor, which is preferably a tube reactor, as specified above, is controlled externally by means of a stream of heat transfer medium which can be, for example, an oil, a salt melt or another liquid capable of transferring heat.

Compared to a synthesis in the liquid phase and compared to a non-isothermal or non-isoperibolic synthesis in the gas phase, the reaction conditions according to the present invention have the advantage of, inter alia, better yields and greater safety in respect of runaway reactions, in particular at high reaction temperatures (e.g. from 200 to 300° C.).

The isothermal or isoperibolic gas phase mode of operation greatly reduces the potential for a runaway reaction during the synthesis. The mass of material present in the reactor which would be available for a runaway reaction is only a fraction of the mass present in a liquid phase process.

The conversion of alcohol is preferably in the range from 80 to 100%, more preferably 99 to 100% or even 99.5 to 100%. The conversion refers to the molar amount of alcohol being consumed in the reaction.

The liquid hourly space velocity is preferably in the range from 0.1 to 2.0 kg, preferably from 0.1 to 1.0 kg, particularly preferably from 0.2 to 0.6 kg, of alcohol per litre of catalyst (bed volume) and hour.

The water formed during the course of the reaction generally does not have any adverse effect on the conversion, the reaction rate, the selectivity and the operating life of the catalyst and is therefore advantageously removed from the reaction product only during work-up of the latter, e.g. by distillation.

The reaction is carried out in the presence of a heterogeneous hydrogenation catalyst. Preferably the catalytically active composition of such catalyst prior to reduction with hydrogen comprises oxygen-containing compounds of copper. More preferably, the catalytically active composition of such catalyst prior to reduction with hydrogen comprises oxygen-containing compounds of copper and another oxidic material which is aluminum oxide, zirconium oxide, titanium oxide and/or silicon dioxide. Preferably the oxidic material is aluminum oxide and/or zirconium oxide and even more preferably aluminum oxide.

For the avoidance of doubt, “and/or” means that the respective oxidic material is any of the listed oxides or a mixture of two or, where applicable, more of the listed oxides.

In a preferred embodiment, the catalytically active composition of the heterogeneous hydrogenation catalyst after its last heat treatment and prior to its reduction with hydrogen comprises:
from 20 to 85% by weight of aluminum oxide (Al2O3), zirconium dioxide (ZrO2), titanium dioxide (TiO2) and/or silicon dioxide (SiO2);
from 1 to 70% by weight of oxygen-containing compounds of copper, calculated as CuO; and
from 0 to 30% by weight, preferably 0 to 25% by weight, particularly preferably 0 to 20% by weight, in particular 0 to 15% by weight of oxygen-containing compounds of nickel, calculated as NiO.

In this context, “and/or” means that the respective catalyst can contain any of the respective oxides as well as any mixture thereof.

The catalytically active composition in such preferred embodiment may further comprise from 0 to 50% by weight of oxygen-containing compounds of magnesium, calculated as MgO, oxygen-containing compounds of chromium, calculated as Cr₂O₃, oxygen-containing compounds of zinc, calculated as ZnO, oxygen-containing compounds of barium, calculated as BaO, and/or oxygen-containing compounds of calcium, calculated as CaO.

In the process of the invention, the catalysts are preferably used in the form of catalysts which consist entirely of catalytically active composition and optionally a shaping aid (e.g. graphite or stearic acid) if the catalyst is to be used as shaped bodies, i.e. contains no further catalytically active accompanying substances.

In this context, the oxidic materials such as titanium dioxide (TiO₂), aluminum oxide (Al₂O₃), zirconium dioxide (ZrO₂) and silicon dioxide (SiO₂) are considered to be part of the catalytically active composition.

The catalysts according to the present invention can for example contain oxygen-containing compounds of nickel or it can be essentially free thereof. For the avoidance of doubt, this also applies to the more preferred, particularly preferred and very particularly preferred embodiments specified below.

In case oxygen-containing compounds of nickel, calculated as NiO, are present, their amount can for example be 5 to 28% by weight, preferably 6 to 20% by weight or particularly preferably 7 to 15% by weight.

It is also possible to use a catalyst, that is essentially free of oxygen-containing compounds of nickel. In such case, the amount of oxygen-containing compounds of nickel, calculated as NiO, is usually less than 5% by weight, preferably less than 1% by weight, particularly preferably less than 0.5% by weight (e.g. less than 0, 1% by weight). An example of such a catalyst is described in DE 10 2004 023 529 A1, paragraphs [0101] and [0102].

To use the catalysts, the catalytically active composition to be milled to powder is introduced into the reaction vessel or the catalytically active composition is installed in the reactor as shaped catalyst bodies after milling, mixing with shaping aids, shaping and heat treatment, for example as pellets, spheres, rings, extrudates (e.g. extruded rods).

The figures (in % by weight) given for the concentrations of the components of the catalyst are, unless indicated otherwise, in each case based on the catalytically active composition of the finished catalyst after its last heat treatment and before it has been reduced by means of hydrogen.

The catalytically active composition of the catalyst after its last heat treatment and before it has been reduced by means of hydrogen is defined as the sum of the catalytically active constituents and the catalyst support materials. In the abovementioned preferred embodiment, the catalytically active composition consists essentially of the following constituents:

Titanium dioxide (TiO₂) and/or aluminum oxide (Al₂O₃) and/or zirconium dioxide (ZrO₂) and/or silicon dioxide (SiO₂) and oxygen-containing compounds of copper and optionally oxygen-containing compounds of magnesium and/or of chromium and/or of zinc and/or of barium and/or of calcium and optionally oxygen-containing compounds of nickel, with the amount of these oxygen-containing compounds of nickel, calculated as NiO, is less than or equal 30% by weight.

The sum of the abovementioned constituents of the catalytically active composition, calculated as Al₂O₃, ZrO₂, TiO₂, SiO₂, CuO, MgO, Cr₂O₃, ZnO, BaO, CaO and NiO, is usually from 70 to 100% by weight, preferably from 80 to 100% by weight, particularly preferably from 90 to 100% by weight, very particularly preferably 100% by weight.

The catalytically active composition of the catalysts used in the process of the invention can further comprise one or more elements (oxidation state 0) or their inorganic or organic compounds selected from groups I A to VI A and I B to VII B and VIII of the Periodic Table.

Examples of such elements and their compounds are:

Transition metals such as Co and CoO, Re and rhenium oxides, Mn and MnO₂, Mo and molybdenum oxides, W and tungsten oxides, Ta and tantalum oxides, Nb and niobium oxides or niobium oxalate, V and vanadium oxides and vanadyl pyrophosphate; lanthanides such as Ce and CeO₂ or Pr and Pr₂O₃; alkali metal oxides such as Na₂O; alkali metal carbonates; alkaline earth metal oxides such as SrO; alkaline earth metal carbonates such as MgCO₃, CaCO₃ and BaCO₃; boron oxide (B₂O₃).

In a more preferred embodiment, the catalytically active composition of the catalysts used in the process of the invention comprises, after its last heat treatment and prior to its reduction with hydrogen,
from 25 to 80% by weight, preferably from 30 to 75% by weight, of aluminum oxide (Al₂O₃), zirconium dioxide (ZrO₂), titanium dioxide (TiO₂) and/or silicon dioxide (SiO₂);
from 2 to 65% by weight, preferably from 5 to 60% by weight, particularly preferably from 20 to 60% by weight, of oxygen-containing compounds of copper, calculated as CuO; and
0 to 30% by weight, preferably 0 to 25% by weight, particularly preferably 0 to 20% by weight, in particular 0 to 15% by weight of oxygen-containing compounds of nickel, calculated as NiO.

In a particularly preferred embodiment, the catalytically active composition of the catalysts used in the process of the invention comprises, after its last heat treatment and prior to its reduction with hydrogen,
from 25 to 80% by weight, preferably from 30 to 75% by weight, of aluminum oxide (Al₂O₃) and/or zirconium dioxide (ZrO₂);
from 2 to 65% by weight, preferably from 5 to 60% by weight, particularly preferably from 20 to 60% by weight, of oxygen-containing compounds of copper, calculated as CuO; and
0 to 30% by weight, preferably 0 to 25% by weight, particularly preferably 0 to 20% by weight, in particular 0 to 15% by weight of oxygen-containing compounds of nickel, calculated as NiO.

In such particularly preferred embodiment, the presence of titanium dioxide (TiO₂) and silicon dioxide (SiO₂) is not excluded. If the catalyst also comprises titanium dioxide (TiO₂) and/or silicon dioxide (SiO₂), the overall amount of aluminum oxide (Al₂O₃), zirconium dioxide (ZrO₂), titanium dioxide (TiO₂) and/or silicon dioxide (SiO₂) amounts preferably to 25 to 80% by weight or 30 to 75% by weight, respectively.

In a very particularly preferred embodiment, the catalytically active composition of the catalysts used in the process of the invention comprises, after its last heat treatment and prior to its reduction with hydrogen,
from 25 to 80% by weight, preferably from 30 to 75% by weight, of aluminum oxide (Al₂O₃);
from 2 to 65% by weight, preferably from 5 to 60% by weight, particularly preferably from 20 to 60% by weight, of oxygen-containing compounds of copper, calculated as CuO; and
0 to 30% by weight, preferably 0 to 25% by weight, e.g. 0 to 20% by weight, in particular 0 to 15% by weight of oxygen-containing compounds of nickel, calculated as NiO.

In such very particularly preferred embodiment, the presence of zirconium dioxide (ZrO₂), titanium dioxide (TiO₂) and silicon dioxide (SiO₂) is not excluded. If the catalyst also comprises zirconium dioxide (ZrO₂), titanium dioxide (TiO₂) and/or silicon dioxide (SiO₂) the overall amount of aluminum oxide (Al₂O₃), zirconium dioxide (ZrO₂) titanium dioxide (TiO₂) and/or silicon dioxide (SiO₂) amounts preferably to 25 to 80% by weight or 30 to 75% by weight, respectively.

In the more preferred, particularly preferred and very particularly preferred embodiment as described above, the catalytically active composition of the catalysts used in the process of the invention may further comprise from 0 to 30% by weight, preferably from 0 to 20% by weight, of oxygen-containing compounds of magnesium, calculated as MgO, and/or oxygen-containing compounds of chromium, calculated as Cr₂O₃, and/or oxygen-containing compounds of zinc, calculated as ZnO, and/or oxygen-containing compounds of barium, calculated as BaO, and/or oxygen-containing compounds of calcium, calculated as CaO.

The oxygen-containing compounds of copper are, in particular, copper(I) oxide and copper(II) oxide, preferably copper(II) oxide.

In a very preferred embodiment, the catalytically active composition of the catalysts, after its last heat treatment and prior to its reduction with hydrogen, essentially consists of
from 25 to 80% by weight, preferably from 30 to 75% by weight, of aluminum oxide (Al₂O₃); and
from 2 to 65% by weight, preferably from 5 to 60% by weight, particularly preferably from 20 to 60% by weight, of oxygen-containing compounds of copper, calculated as CuO.

In another very preferred embodiment, the catalytically active composition of the catalyst, after its last heat treatment and prior to its reduction with hydrogen, essentially consists of
from 25 to 80% by weight, preferably from 30 to 75% by weight, of aluminium oxide (Al₂O₃),
from 2 to 65% by weight, preferably from 5 to 60% by weight, particularly preferably from 20 to 60% by weight, of oxygen-containing compounds of copper, calculated as CuO; and
less than 30% by weight, preferably. 5-28% by weight, more preferably less 6 to 20% by weight, even more preferably 7 to 15% by weight, of oxygen-containing compounds of nickel, calculated as NiO.

The term “essentially consist of” means that the catalytically active composition of the catalyst consists of more than 95% by weight, preferably 99% by weight of aluminium oxide, oxygen-containing compounds of copper and where applicable oxygen-containing compounds of nickel.

The catalysts used in the process of the invention can be prepared by various methods. For such methods reference is made to DE 10 2004 023 529 A1, in particular paragraphs [0046] to [0063], which are herewith incorporated by reference.

The process according to the invention is particularly suited for the production of respective cyclic amines, the process comprises reaction of a diol with ammonia.

Suitable diols are primary aliphatic diols having 2 to 6, preferably 4 to 6, carbon atoms. In this context, the term “aliphatic” shall mean any functionalized or unfunctionalized organic residue that contains no aromatic ring system and is not cyclic. It can have any functional group including any heteroatoms, e.g. oxygen.

Preferred aliphatic diols are selected from the group consisting of diethylene glycol, 1,4-butandiol, 1,5-pentanediol and 1,6-hexanediol. 1,4-butanediol is particularly preferred.

Within the reaction of such diols with ammonia, additional value products may occur depending on the respective reaction conditions.

The process according to the present invention is also particularly suited for the production of methoxy-2-propylamine, the process comprises reacting 1-methoxy-2-propanol with ammonia.

In addition, the process according to the present invention is also particularly suited for the production of mono-, di- and trihexylamine, the process comprises reacting hexane-1-ol with ammonia.

The process according to the invention is more particularly suited for the coproduction of pyrrolidine and bis(pyrrolidino)butane, the process comprises reacting 1,4-butanediol with ammonia.

Without wanting to be bound by any theory or limiting the scope of the present invention in whatsoever kind, it is believed that the coproduction of pyrrolidine and bis(pyrrolidino)butane occurs via the following reaction scheme.

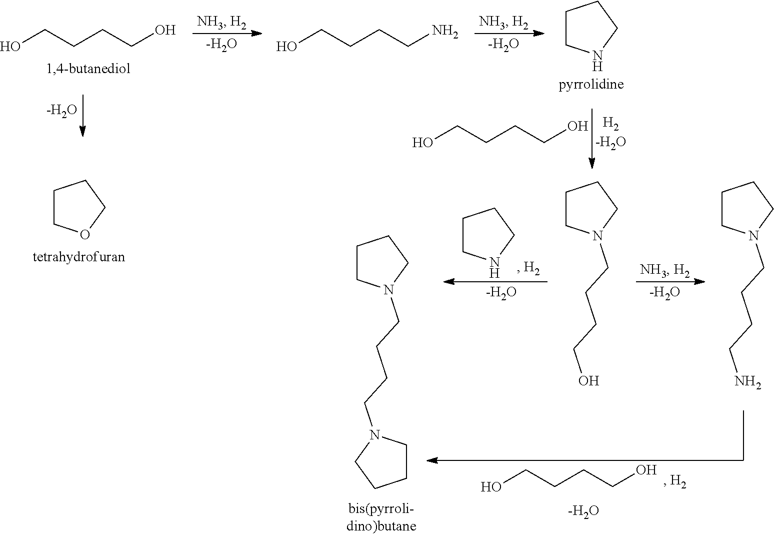

The ratio of pyrrolidine and bis(pyrrolidino)butane can be varied depending on the reaction conditions and the process set-up. Operating the pressure separator within the temperature ranges according to the invention increases the yield of economically more favorable pyrrolidine.t

Instead of or together with recycling pyrrolidine it is also possible to feed fresh pyrrolidine together with ammonia and alcohol into the reactor.

In case of the coproduction of pyrrolidine and bis(pyrrolidino)butane, the product stream obtained from the pressure separator inter alia contains pyrrolidine, bis(pyrrolidino)butane, 4-hydroxybutylpyrrolidine, 4-aminobutylpyrrolidine, high boilers with a boiling point higher than that of bis(pyrrolidinyl)butane, ammonia and water. Preferably such product stream is further separated, e.g. by distillation. It is principally possible to recycle the pyrrolidine thus removed from the product stream to the reaction which leads to an increase of bis(pyrrolidino)butane production. Without wanting to be bound by any theory or limiting the scope of the present invention in whatsoever kind, it is believed that such recycle increases the pyrrolidine concentration in the reactor and thereby favoring the reaction towards bis(pyrrolidino)butane as can be seen from the reaction scheme presented above.

FIG. 1 represents an embodiment that is particularly preferred. Alcohol via line (1) and recycle gas via line (2) are fed into evaporator (4). The recycle gas is passed through compressor (11) in order to increase its pressure to the desired reaction pressure. Fresh hydrogen and ammonia can be fed directly into the evaporator (4) via lines (3a) and (3a′) or they can be fed into the recycle gas via lines (3b) and (3b′). They can also be fed into the recycle gas before the latter is passed through the compressor (11) via lines (3c) and (3c′). This is advantageous because both the hydrogen and the ammonia stream have a higher pressure than the recycle gas, thus reducing the amount of energy required by the compressor. Hydrogen and ammonia can also be fed directly into the reactor via lines (3d) and (3d′). Theoretically, a combination of any such ways to add hydrogen (i.e. (3a) to (3d)) and ammonia (i.e. (3a′) to (3d′)) is also possible. In the evaporator (4) the alcohol is evaporated, and the resulting gaseous stream is fed into the reactor (6) via line (5). The reaction mixture is passed through heat exchanger (8) and optionally through a cryostat (not shown in FIG. 1) via line (7), where it is cooled down and fed into pressure separator (9) where a gaseous stream, consisting essentially of hydrogen and ammonia, is withdrawn. Via line (10) said gaseous stream is partly discharged. The remainder is recycled via line (2) to evaporator (4) as the recycle gas stream.

It is further possible to feed the crude reaction product from pressure separator (9) via line (12) into low-pressure separator (13) where it is further degassed. The resulting gaseous stream, consisting essentially of hydrogen, ammonia and respective low boilers, is discharged via line (15). The crude reaction product, in particular value product amine and high boilers are withdrawn form low-pressure separator (13) via line (14). Said crude amine product can be subjected to further purification.

It is also possible to feed the crude reaction product from pressure separator (9) via line (12) into a distillation column (not shown in FIG. 1), where hydrogen, ammonia and respective low boilers are removed. Preferably, the ammonia is recycled to the reaction as fresh ammonia.

Usually a low-pressure separator is used on a laboratory scale and a respective distillation column on an industrial scale.

The following examples only serve for the purpose of the illustration of the present invention and shall therefore not limit it in whatsoever kind.

### EXAMPLES

### Catalyst

The following examples were carried out using a copper/nickel catalyst having the composition 45% by weight of CuO and 10% by weight of NiO, the remainder up to 100% is gamma-Al₂O₃ (after its last heat treatment and before reduction with hydrogen).

The catalyst was prepared according to Example 1 of DE-A-24 45 303. Before commencement of the reaction, the catalyst was reduced (see below).

### Examples 1 to 4

The experiments were carried out continuously in gas phase furnace reactors through which the reactants flowed from the bottom upward in a 2.1 m long oil-heated double-walled tube which had an internal diameter of 4.8 cm and was filled from the bottom upward with 40 ml of ceramic spheres (2.5-3.5 mm), 1 liter of catalyst and 1.5 liters of inert material (ceramic spheres, 2.5-3.5 mm). The reactor was operated at 20 bar. The shaped catalyst bodies in pellet form were used in sizes of 5×5 mm (i.e. 5 mm diameter and 5 mm height). After installation in the reactor, all catalysts were activated at atmospheric pressure according to the following method: 12 h at 180° C. (oil circuit reactor) with 20 NL/h and 400 NL/h of N2, 12 hat 200° C. with 20 NL/h of 40 H2 and 400 NL/h of N2, replace N2 by 200 NL/h of H2 over 6 h, 6 h at 240° C. with 200 NL/h of H2. (NL=standard liters=volume converted to STP). The feed streams fresh hydrogen, circulating gas, pressurized gases and starting materials were heated to the desired reactor temperature by means of a system comprising three coil heat exchangers. The third heat exchanger was regulated via a temperature sensor just before the reactor. The oil heating of the double-wall reactor was likewise set to the desired reactor temperature. By means of two further coil heat exchangers, the reactor output was cooled firstly with river water and then heated to the desired temperature of the pressure separator (25 to 49° C.) using a cryostat and was fed to a pressure separator. The pressure separator was operated at a pressure of about 2.5 bar below the reaction pressure (20 bar). The separation of liquid phase and gas phase occurred there. The liquid phase was depressurized in a low-pressure separator maintained at 45° C. from where the released gases were discharged via the offgas and the liquid was conveyed into the output drum. The gas phase from the pressure separator was recirculated in a defined amount via a circulating gas compressor and once again served as carrier gas for the starting materials. A pressure regulator ensured that excess gas was conveyed to the muffle furnace for incineration.

Conversion and selectivity of the output were determined by gas-chromatographic analysis and are reported in corrected GC area %.

The set-up as described above corresponds to the set-up described in FIG. 1

The reaction conditions for Examples 1 to 4 were as follows:
Reactor inlet temperature: 199° C.
Reactor outlet temperature: 209° C.
Evaporator temperature: 220° C.
Reaction pressure: 20 bar
Liquid hourly space velocity: 0.5 kg BDO/(L (Cat.)·h)
Molar Ratio (fresh NH₃:BDO): 3:1
Hydrogen flow: 150 NL/[L of catalyst (bed volume)]·h]
Recycle gas flow rate: 7 Nm³/[L of catalyst (bed volume)]·h]
Conversion BDO: 100%
(Nm³=standard cubic meters=volume converted to STP)

The results are presented in table 1 below.

TABLE 1
Results

| | Temp. | | | 4-Hydroxy/-4- | | |
|---|---|---|---|---|---|---|
| | pressure | | | Aminobutyl- | Alkyl- | Bis(pyrrolidino)- |
| | separator | Pyrrolidine | THF | pyrrolidine | pyrrolidines | butane |
| # | [° C.] | [%] | [%] | [%] | [%] | [%] |
| 1 | 25 | 79.0 | 0.9 | 1.5 | 2.8 | 14.6 |
| 2 | 35 | 83.6 | 0.7 | 1.4 | 2.5 | 10.9 |
| 3 | 45 | 83.5 | 0.6 | 1.6 | 2.4 | 10.9 |
| 4 | 49 | 83.2 | 0.5 | 1.7 | 2.3 | 11.0 |

### Discussion of Results

According to the results presented in table 1, an increase of pyrrolidine selectivity can be achieved by an increase of the temperature in the pressure separator. Within the temperature range from 25 to 35° C. a plateau is reached. Furthermore, the overall selectivity towards amine products (in particular value products pyrrolidine and bis(pyrrolidino)butane) is increased. Moreover, the amount of the unwanted by-product THF is constantly reduced.

1.-15. (canceled)

16. A process for the continuous production of amines, the process comprising reacting a primary or secondary alcohol with ammonia in the presence of hydrogen and a heterogeneous hydrogenation catalyst in the gas phase using a recycle gas mode, wherein the temperature in the pressure separator is greater than 20° C.

17. The process according to claim 16, wherein the temperature in the pressure separator is greater than 21° C.

18. The process according to claim 16, wherein the pressure separator is operated at a pressure close to the reaction pressure.

19. The process according to claim 18, wherein the pressure in the pressure separator is 0.01 to 10 bar below the reaction pressure.

20. The process according to claim 16, wherein the recycle gas stream has a flow rate in the range from 40 to 1500 m³ at operating pressure /[m³ of catalyst (bed volume)·h].

21. The process according to claim 16, wherein fresh ammonia is added in a molar amount which is from 0.90 to 100 times that of the alcohol.

22. The process according to claim 16, wherein the reaction is carried out at an absolute pressure in a range from 1 to 300 bar.

23. The process according to claim 16, wherein the reaction is carried out at a temperature in a range from 80 to 300° C.

24. The process according to claim 16, wherein the liquid hourly space velocity is in the range from 0.1 to 2.0 kg of alcohol per litre of catalyst bed volume an hour.

25. The process according to claim 16, wherein the catalytically active composition of the heterogeneous hydrogenation catalyst after its last heat treatment and prior to its reduction with hydrogen comprises:
from 20 to 85% by weight of aluminum oxide (Al2O3), zirconium dioxide (ZrO2), titanium dioxide (TiO2) and/or silicon dioxide (SiO2);
from 1 to 70% by weight of oxygen-containing compounds of copper, calculated as CuO; and
from 0 to 30% by weight of oxygen-containing compounds of nickel, calculated as NiO.

26. The process according to claim 16, wherein the catalytically active composition of the heterogeneous hydrogenation catalyst after its last heat treatment and prior to its reduction with hydrogen comprises:
from 25 to 80% by weight, of aluminum oxide (Al₂O₃) and/or zirconium dioxide (ZrO₂);
from 2 to 65% by weight, of oxygen-containing compounds of copper, calculated as CuO; and
0 to 30% by weight of oxygen-containing compounds of nickel, calculated as NiO.

27. The process according to claim 16, wherein the catalytically active composition of the heterogeneous hydrogenation catalyst after its last heat treatment and prior to its reduction with hydrogen comprises:
from 25 to 80% by weight of aluminum oxide (Al₂O₃);
from 2 to 65% by weight of oxygen-containing compounds of copper, calculated as CuO; and
0 to 30% by weight of oxygen-containing compounds of nickel, calculated as NiO.

28. The process according to claim 25, wherein the catalytically active composition of the heterogeneous hydrogenation catalyst after its last heat treatment and prior to its reduction with hydrogen comprises 5 to 28% by weight of oxygen-containing compounds of nickel, calculated as NiO.

29. The process according to claim 16 for the production of
respective cyclic amines, the process comprises reacting a diol,
1-methoxy-2-propylamine, the process comprises reacting 1-methoxy-2-propanol with ammonia, and
mono-, di- and trihexylamine, the process comprises reacting hexane-1-ol with ammonia.

30. The process according to claim 16 for the coproduction of pyrrolidine and bis(pyrrolidino)butane, the process comprises reacting 1,4-butanediol with ammonia.
